(19) United States
(12) Patent Application Publication
(10) Pub. No.: US 2009/0098617 A1
(43) Pub. Date: Apr. 16, 2009

(54) ENZYMATIC TREATMENT UNDER VACUUM OF LIGNOCELLULOSIC MATERIALS

(76) Inventors: Murray Burke, Oakville (CA); Bradley Saville, Oakville (CA); Claudia Ishizawa, Houston, TX (US)

Correspondence Address: BERESKIN AND PARR, 40 KING STREET WEST, BOX 401, TORONTO, ON, M5H 3Y2, CA

(21) Appl. No.: 12/249,330

(22) Filed: Oct. 10, 2008

Related U.S. Application Data
(60) Provisional application No. 60/978,795, filed on Oct. 10, 2007.

(51) Int. Cl. C12P 19/14; C12P 7/02

(52) U.S. Cl. 435/99; 435/155

(57) ABSTRACT

A method for treating plant materials to release fermentable sugars is disclosed. More specifically, an enzymatic hydrolysis process for treating lignocellulosic materials performed under vacuum and producing a sugar rich process stream that may subsequently be subjected to fermentation to produce biofuels and chemicals is disclosed.

### CROSS-REFERENCE

This application is a regular application claiming priority from U.S. Provisional application 60/978,795 filed on Oct. 10, 2007.

### FIELD

This application relates to a method for treating plant materials to release fermentable sugars. More specifically, this application relates to an enzymatic hydrolysis process for treating lignocellulosic materials performed under vacuum and producing a sugar rich process stream that may subsequently be subjected to fermentation to produce biofuels and chemicals.

### BACKGROUND

Although biomass has long shown promise as a renewable source of fuel energy, there remains a need for more efficient means of transforming biomass into suitable biofuels. Plant materials are a significant source of fermentable sugars, such as glucose that can be transformed into biofuels. However, the sugars in plant materials are contained in long polymeric chains of cellulose and hemicellulose. Utilizing current fermentation processes, it is necessary to break down these polymeric chains, such as into monomeric sugars, prior to the fermenting step.

Methods of converting plant biomass into fermentable sugars are known in the art and in general, comprise two main steps: a pretreatment step to loosen the plant structure, and an enzymatic or chemical hydrolysis step to convert the polymeric chains of cellulose and hemicellulose into monomeric sugars. Several approaches have been used for the pretreatment step, e.g., autohydrolysis, acid hydrolysis, ammonia activation, kraft pulping, organic solvent pulping, hot water pretreatment, ammonia percolation, lime pretreatment, caustic solvent pulping, or alkali peroxide pretreatment. Each pretreatment technology has a different mechanism of action on the plant structure, inducing either physical and/or chemical modifications. However, the main objective of the pretreatment is to provide accessibility of the plant material to the enzymes. In the autohydrolysis process, the acetyl groups attached to hemicelluloses are broken down by steam and pressure releasing organic acids, e.g., acetic acid, giving the conditions for a mild acid hydrolysis process. Although a simple process, the yield of fermentable sugars is poor, in addition to the process requiring a significant amount of energy.

Jakobsen et al. (U.S. Pat. No. 5,874,274) discloses the use of a single step enzymatic process for reducing the viscosity of a plant material using xylanase and cellulase, and in particular for the treatment of wheat.

### SUMMARY

This application relates to an enzymatic process performed under vacuum to prepare a sugar rich process stream from a feedstock derived from plant materials. Preferably, a two-stage enzymatic process is used, in which case vacuum may be applied in any stage. However, vacuum is preferably applied in at least the first stage. The process and apparatus may result in the conversion of at least 60%, preferably more than 75% and more preferably over 90% of the cellulose and hemicelluloses to monomeric sugars. The sugar rich process stream may subsequently be subjected to fermentation to produce an alcohol stream, which may also be performed under vacuum. The alcohol stream from the fermentation stage (i.e., the raw alcohol stream) may have an ethanol content of about 3 to about 22% v/v. Optional operating ranges include about, 5 to about 15% and preferably about 5 to about 22% as well as about 8 to about 12%, preferably about 8 to about 15% and more preferably about 8 to about 22%. Such alcohol concentrations may be obtained without using corn as a feedstock.

Cellulosic ethanol processes, namely processes that produce ethanol from sugars obtained by breaking down the cellulose and/or hemicellulose from non-corn plant fiber (i.e. plant fiber that excludes corn kernels), typically produce a raw alcohol stream having an ethanol content of about 2-6% v/v. With the process and apparatus described in this application, cellulose ethanol plants may produce a raw alcohol stream having a comparable alcohol concentration to that obtained by corn based ethanol plants, namely plants that produce ethanol from sugars obtained from the starch in corn. Accordingly, one advantage of the process and apparatus of this invention is that the amount of water to be removed from the raw alcohol stream to produce a fuel ethanol stream having a comparable concentration to the concentration of a product stream from a corn based ethanol plant is substantially reduced compared to current cellulosic ethanol plant technology. As a fuel ethanol stream is typically produced by distillation, the process and apparatus described here therefore results in a substantial reduction in energy required for the distillation process and, optionally, a substantial reduction in the size (i.e., the diameter) of the distillation column compared to current cellulose ethanol plant technology. Furthermore, the processes of the present invention allow for a higher solid concentration (lignocellulosic feedstock) to begin in the enzymatic processes. Consequently, as the solid concentration increases, the sugar concentration also increases, resulting in a lower fermentation volume, which represents a 2 to 3 times reduction when compared to current cellulosic ethanol plant technology.

In one embodiment, the feedstock is subjected to an enzymatic hydrolysis process under vacuum. The enzymatic hydrolysis produces a volatile component stream, which is removed by the vacuum. The compounds in the volatile component stream may be produced upstream of the enzymatic hydrolysis process and/or during the enzymatic hydrolysis process. The compounds have an inhibitory effect on one or more of the enzymes used in the process. Accordingly, an advantage of the invention is that the enzymatic hydrolysis process may proceed further towards complete treatment of the feedstock as the enzymatic activity is not subject to a high level of inhibitory compounds.

In an embodiment of the disclosure, the enzymatic hydrolysis is performed under a slight vacuum. The vacuum may be from 700 to 50 mm Hg (i.e, the pressure in the vessel may be from 700 to 50 mm Hg). Preferably, the vacuum is less than about 600 mm Hg, more preferably less than about 100 mm Hg and most preferably less than about 50 mm Hg. Preferably, the maximum vacuum that is applied is about 4 mm Hg.

In a preferred alternate embodiment, the feedstock is subjected to a first enzymatic hydrolysis process to reduce the viscosity of the feedstock and produce a low viscosity effluent stream. In an embodiment, the viscosity of the low viscosity effluent stream is at least about 15% lower than the initial feedstock slurry, preferably at least about 20% lower, preferably at least about 50% lower, more preferably at least about 66% lower and most preferably at least about 90% lower. During the first enzymatic hydrolysis, hemicellulose and cellulose are broken down, preferably to soluble oligosaccharides of sugars. During this step, it is preferred to preferentially hydrolyze the hemicelluloses instead of the celluloses (e.g., preferentially acts on the hemicellulose relative to the cellobiose in the feedstock). For example, this process step may utilize an enzyme preparation comprising hemicellulase and cellulase activities. While it will be appreciated that a suitable enzyme preparation will typically contain enzymes that may act on the cellulose, it is preferred that only a portion of the hemicelluloses will be converted.

Subsequently, if a two-stage process is used, the product stream from the first enzymatic hydrolysis process, which has a lower viscosity, is subjected to a second enzymatic hydrolysis process, which is optionally also performed under vacuum. The second enzymatic hydrolysis process preferably utilizes enzymes to hydrolyze cellulose as well as to convert the oligosaccharides to monomeric sugars suitable for fermentation. Preferably, this second enzyme preparation comprises beta-glucosidase activities. For example, the second enzyme preparation may have an activity to convert cellulose and cellobiose to monomers and cello-oligosaccharides. In this second enzymatic hydrolysis process, it is preferred that all, or essentially all, (e.g., preferably at least 60, more preferably at least 75 and most preferably at least 90%) of the remaining cellulose and hemicelluloses, and their oligosaccharides, are converted, to the extent desired, but preferably to the extent commercially feasible, to monomeric sugars.

Without being limited by theory, oligosaccharides, and in particular cellobiose, have an inhibitory effect on cellulase enzymes and, in particular, on endo-gluconases and cellobiohydrolases, Accordingly, in a first step, the hemicelluloses, and optionally the cellulose, are treated with enzymes to produce soluble sugars. However, the process is conducted so as not to render a substantial portion of the cellulose into monomers or dimers, such as cellobiose. While it will be appreciated that enzymatic hydrolysis will result in the production of some monomers and cellobiose, the process is conducted so as to prevent a substantial inhibition of the enzymes. Subsequently, in a second enzymatic process, the oligosaccharides are subjected to enzymatic hydrolysis to produce fermentable sugars (preferably monomers).

Preferably, the first enzyme preparation preferentially acts on the hemicellulose. In accordance with this embodiment, without being limited by theory, it is believed that in such a first enzymatic process, the hemicellulose is broken down into oligomers and monomers that are removed from the fiber as soluble compounds in an aqueous medium (preferably water). This targeted enzymatic process opens up the fiber structure by the breakdown of the hemicellulose and the removal of the lower molecular weight compounds. In this application, the term preferentially hydrolyze means that a significant portion of the enzymes that are used target the hemicelluloses instead of the celluloses, even though some of the enzymes present may still target the celluloses. Preferred preferential hydrolysis in the first stage, include hydrolyzing about 60% or more, and preferably about 85% or more, of the hemicelluloses while preferably, hydrolyzing less than about 25, and more preferably less than about 15% of the celluloses. The resultant more open fiber structure permits enzymes, such as cellulases, to more readily enter the fiber structure and hydrolyze the cellulose. Accordingly, the second enzymatic hydrolysis step uses enzymes that preferentially target cellulose relative to hemicellulose in the feedstock (e.g., the second enzyme preparation preferentially acts on the cellulose and cellobiose relative to xylans in the feedstock). It will be appreciated that the second enzymatic hydrolysis step may use an enzyme preparation that includes enzymes that target hemicelluloses. However, as most of the hemicelluloses may have already been treated in the first stage, a relatively large percentage of such enzymes may not be required in the second enzyme preparation.

Without being limited by theory, it is believed that during the first enzymatic hydrolysis stage, xylan is converted to soluble xylan (soluble oligomers), and to a degree xylose, and mannan is converted to mannose. The first enzyme preparation preferentially acts upon the β-1,4 linkage of the xylose residues of xylan and the β-1,4 linkage of the mannose residues of mannan. These rates of reaction strongly parallel the viscosity reduction that is produced by this stage. Accordingly, it is believed that the enzymatic hydrolysis of the hemicellulose results, at least in part, in the viscosity reduction and may be the main factor in the viscosity reduction.

However, many commercial hemicellulase enzyme preparations also possess cellulase activity, which may also contribute to the viscosity reduction. In particular, as the hemicellulose is hydrolyzed, water is released from the fiber, in addition to the production of oligosaccharides and monomeric sugars. Moreover, this hydrolysis results in the reduction in the length of hemicellulose and cellulose polymer chains. The release of water and the reduction in molecular chain length may also be a factor, or a key factor in the rapid decrease in viscosity of the mixture in the reactor during the first stage of enzymatic hydrolysis.

During the enzymatic hydrolysis processes, acetyl groups are removed from the hemicellulose. In an aqueous medium, these form acetic acid. Acetic acid reduces the pH of the mixture in the reactor, e.g., from about 4.9 to about 4.4. This pH reduction has an inhibitory effect on the first stage enzyme preparation. Therefore, preferably, at least a portion of the acetic acid and/or other inhibitory compounds, such as furfural, are volatilized and removed from the process. In addition, some of the acetic acid may also be neutralized by the addition of a neutralizing agent (e.g., urea, anhydrous ammonia, aqueous ammonia, sodium hydroxide, potassium hydroxide). As acetic acid is relatively volatile, it may be drawn off by vacuum as it is produced. Further, as the enzymatic process reduces the viscosity of the mixture in the reactor, the mixture is more easily inducted to flow, e.g., due to stirring, and the acetic acid has a greater chance to reach the surface of the mixture and volatilize.

In an embodiment of the disclosure, at least a portion of the volatile inhibitory compounds, such as acetic acid, furfural and hydroxymethylfurfural are removed from the mixture that is subjected to enzymatic hydrolysis. Other compounds and/or molecules that are also removed include nitrogen, oxygen, argon and carbon dioxide. Preferably, a sufficient vacuum is provided for a sufficient amount of time so as to reduce or maintain a concentration of acetic acid in the mixture that is less than 0.4% (w/v), preferably less than 0.3% and most preferably less than 0.2%. Alternately, or in addition, a sufficient vacuum is provided for a sufficient amount of time so as to reduce or maintain a concentration of furfural in the mixture that is less than 0.2% (w/v), preferably less than 0.1% and most preferably less than 0.05%. It will be appreciated that the greater the vacuum, the more of the volatile inhibitory compounds that will be removed. Preferably a generally constant vacuum is applied. Preferably, the vacuum is applied during the entire time that the enzymatic hydrolysis is conducted.

Prior to the first enzymatic hydrolysis, the feedstock is optionally subjected to an activation step, and in an embodiment, the activation step is an autohydrolysis process in a digester. During the autohydrolysis, the feedstock is subjected to a high temperature and a high pressure in a digester, which is believed to activate the polymeric structure of cellulose and hemicellulose by introducing water molecules between the polymeric chains. In addition, during autohydrolysis, the degree of polymerization of cellulose and hemicellulose may be reduced from about 10,000 to about 1,500-1,000. Also during autohydrolysis, volatile compounds such as acetic acid are released from the feedstock.

In another embodiment, after autohydrolysis, the feedstock is transferred from the autohydrolyzer to a solid/vapor separation unit. In one embodiment, the solid/vapor separation unit is a cyclone, preferably under a vacuum. The difference in pressure from the high pressure autohydrolyzer to the low pressure cyclone results in separation of solids from volatile compounds. The volatile compounds, which may include some volatile inhibitory compounds that are present in the mixture prior to enzymatic hydrolysis, are removed from the cyclone by the vacuum. In addition, it is believed that the low pressure of the cyclone opens up the fiber structure of the cellulose and hemicellulose, increasing the surface area of the fiber, which allows greater access for the enzymes.

Further aspects and advantages of the embodiments described herein will appear from the following description taken together with the accompanying drawing.

### BRIEF DESCRIPTION OF THE DRAWINGS

For a better understanding of the embodiments described herein and to show more clearly how they may be carried into effect, reference will now be made, by way of example only, to the accompanying drawing which shows at least one exemplary embodiment, and in which:

FIG. 1 is a flow chart of the method according to the preferred embodiment that includes optional steps;

FIG. 2 is a flow chart of the method according to another embodiment that shows additional details regarding specific process steps;

FIG. 3 is a graph demonstrating the amount of water recovered from a pre-treated poplar held under vacuum at varying temperatures and pH in accordance with another embodiment of the disclosure;

FIG. 4 is a graph demonstrating the amount of furfural recovered from a pre-treated poplar held under vacuum at varying temperatures and pH in accordance with another embodiment of the disclosure; and,

FIG. 5 is a graph demonstrating the amount of acetic acid recovered from a pre-treated poplar held under vacuum at varying temperatures and pH in accordance with another embodiment of the disclosure.

### DETAILED DESCRIPTION

This application relates generally to a method of treating a lignocellulosic feedstock to breakdown cellulose and hemicellulose in the feedstock into monomeric sugars such as glucose, which may be fermented to produce alcohol. In particular, this application relates generally to the use of enzymatic hydrolysis under vacuum. The applicants have surprisingly found that subjecting the lignocellulosic feedstock to an enzymatic hydrolysis process under vacuum and removing a volatile components stream from the feedstock improves the yield of fermentable sugars and/or the purity of the resulting sugar rich process stream. Accordingly, the use of enzymatic hydrolysis operated under vacuum allows for a reduction in the viscosity of the feedstock and the production of a process stream that is rich in fermentable sugars.

In an optional embodiment, the applicants have found that activating and/or physically modifying the feedstock prior to the enzymatic hydrolysis process results in an increased yield of fermentable sugars in the process stream and/or a faster reaction rate.

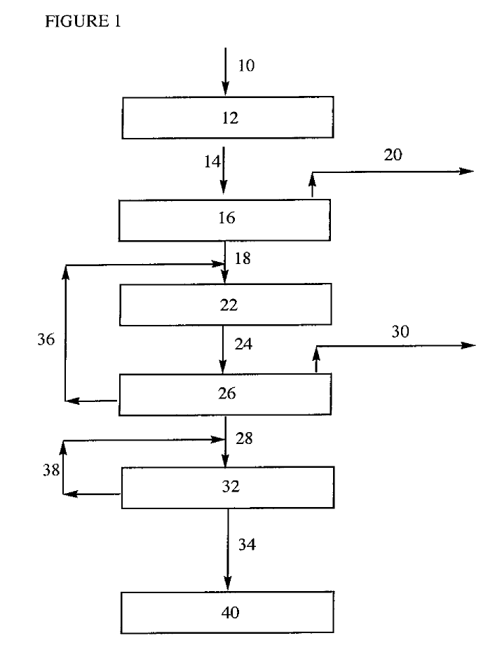

FIG. 1 exemplifies a schematic of one embodiment of the invention utilizing a two stage enzymatic hydrolysis process. It will be appreciated that the invention relates to a process using one or more enzymatic hydrolysis stages. Each enzymatic hydrolysis reactor, or only one of them, may be operated under vacuum. If there are a plurality of enzymatic hydrolysis stages or reactors that are operated in series, then at least the first stage or reactor is preferably operated under vacuum.

The lignocellulosic feedstock 10 is optionally first subjected to activation, extraction, hydrolysis and/or physicochemical modification step 12 such as by autohydrolysis to produce an activated feedstock stream 14. The activated feedstock stream 14 is then optionally transferred to a solid/vapour separator, such as cyclone 16, which separates the solid activated feedstock from volatile compounds. Discharging the charge from the high pressure autohydrolysis digester 12 to, e.g., cyclone 16, results in a rapid release of pressure and causes physical modification of the fibrous material via steam explosion. As the cyclone is operated at vacuum pressure, volatile compounds are removed by the vacuum in a volatile compound stream 20. The solid activated feedstock stream 18 is then optionally fed to a disc refiner 22 to produce a fine particulate stream 24. It will be appreciated that neither of these optional steps, or one or both of these optional steps may be utilized.

Fine particulate stream 24 may then subjected to enzymatic hydrolysis, and preferably a two-stage enzymatic hydrolysis process. If a two stage enzymatic hydrolysis is used, then the first enzymatic hydrolysis stage 26 is preferably operated under vacuum and produces a low-viscosity effluent stream 28 and a volatile components stream 30, which is removed from the reactor. The low viscosity effluent stream 25 may then be subject to a second enzymatic hydrolysis stage 32, which is optionally performed under vacuum, to produce a sugar rich process stream 34.

All or a portion of the material subjected to a first enzymatic hydrolysis step is preferably reprocessed by recycle stream 36 and returned to the reactor 26, preferably by at least a portion of, and preferably all of, the recycle stream being passed through disc refiner 22 before being reintroduced to the first enzymatic hydrolysis stage 26. The recycle stream may be mixed with fresh lignocellulosic feedstock as exemplified prior to being introduced into the disc refiner 22. It will be appreciated that some or all of the recycle stream may be fed directly into reactor 26.

It will also be appreciated that all or a portion of the material being subjected to the second enzymatic hydrolysis step 32 is preferably removed by recycle stream 38 and returned to the reactor 32.

The sugar rich process stream 34 may then be subjected to further processing, preferably including a fermentation step 40 to produce ethanol, or it may be stored or used in other chemical processes. Fermentation step 40 is also preferably performed under to vacuum.

### Input Feedstock

The lignocellulosic feedstock is derived from plant materials. As used herein, a “lignocellulosic feedstock” refers to a plant fiber containing cellulose, hemicellulose and lignin. The applicants contemplate other sources of plant materials comprising cellulose, hemicellulose and lignin for use in deriving lignocellulosic feedstocks and any of those may be used. In some embodiments, the feedstock may be derived from trees, preferably deciduous trees such as poplar (e.g. wood chips). Alternately or in addition, the feedstock may also be derived from agricultural residues such as corn stover, wheat straw, barley straw, rice straw, switchgrass, sorghum, sugarcane bagasse, rice hulls and/or corn cobs. Preferably, the lignocellulosic feedstock comprises agricultural residues and wood biomass, more preferably wood biomass and most preferably deciduous. Accordingly, the feedstock may be any feedstock that does not contain edible agricultural produce, however such material may be used.

The lignocellulosic feedstock is preferably cleaned, e.g., to remove ash, silica, metal strapping (e.g., from agricultural products), stones and dirt. The size of the components of the lignocellulosic feedstock may also be reduced. The size of the components of the feedstock may be from about 0.05 to about 2 inches, preferably from about 0.1 to about 1 inch, and more preferably from about 0.125 to about 0.5 inches in length.

It will be appreciated that if the optional activation, extraction, hydrolysis or physical modification is not utilized, the feedstock may be further crushed, ground or otherwise modified so as to decrease the average particle size and increase the surface area of the material in the feedstock. Accordingly, the size of the components of the feedstock may be from about 0.0625 to about 2 inches, preferably from about 0.125 to about 1 inch and more preferably from about 0.125 to about 0.5 inches. Any process machinery that is able to crush, grind or otherwise decrease the particle size may be utilized. The feedstock that is fed to the optional disc refiner preferably comprises from 1% to 60% wt total solids

### Activation

The lignocellulosic feedstock is optionally subjected to one or more activation steps prior to the feedstock being subject to enzymatic hydrolysis. As used herein an “activated” feedstock refers to a feedstock that has been treated so as to increase the susceptibility of cellulose and hemicellulose in the feedstock to subsequent enzymatic hydrolysis. In addition, the lignocellulosic feedstock may also be subjected to chemical or physical modification pretreatment, extraction or hydrolysis.

The applicants have found that certain processes for treating lignocellulosic feedstocks are surprisingly beneficial for preparing the feedstocks for enzymatic hydrolysis. Without being limited by theory, the applicants believe that activation involves the chemical activation of hydrogen bond sites in the hemicellulose and cellulose polymer chains.

Methods of activation, extraction, hydrolysis, and chemical or physical modification include, but are not limited to, autohydrolysis, acid-hydrolysis, ammonia activation, disc refining, kraft pulping, organic solvent pulping, hot water pretreatment, ammonia percolation, lime pretreatment, caustic solvent pulping and alkali peroxide pretreatment. Any process equipment known in the art may be used. Preferably, at least one of disc refining and autohydrolysis is utilized and more preferably, both are utilized.

In some embodiments, the feedstock is subjected to autohydrolysis. Autohydrolysis is a process of breaking down hemicellulose and cellulose by exposure to high temperatures, steam and pressure, preferably in the presence of a chemical agent, such as sulphuric acid. When performed in the presence of an acid, an autohydrolysis process is known as an acid hydrolysis. Autohydrolysis often results in the release of acetic acid from the breakdown of acetylated hemicellulose, which further helps the hydrolysis process.

Preferably, the autohydrolysis is conducted in a steam explosion digester, which is known in the art. For example, feedstock having a moisture content of about 45 to about 55% by weight may be fed to an autohydrolysis digester wherein the biomass is hydrolyzed under steam at high pressure (e.g. 100-400 psig) and temperature (e.g., 150-2500° C.), optionally in the presence of a catalyst, such as sulphuric acid. In autohydrolysis, the acetyl groups are hydrolyzed from the plant structure producing acetic acid. The release of acetic acid decreases the pH of the reaction mixture in the digester from, e.g., neutral, to acidic (e.g., 3.0-4.0) supplying acid conditions for a mild acid hydrolysis reaction. During the autohydrolysis step, hemicellulose is partially hydrolyzed to xylose, soluble xylo-oligosaccharides and other pentosans. The yield may be up to about 75%.

During autohydrolysis, the degree of polymerization of cellulose and hemicellulose may be reduced from about 10,000 to about 1,500-1,000. This process is preferably carried out above the glass transition temperature of lignin (120-160° C.). Depending upon the severity of the reaction, degradation products may still be produced, such as furfural, hydroxyl-methylfurfural, formic acid, levulinic acid and other organic compounds.

At the instant of release from the digester (steam explosion), the biomass exits the high temperature, high pressure hydrolyzer into a reduced pressure cyclone, preferably atmospheric pressure and, more preferably into a vacuum. The pressure in the digester is suddenly released, e.g., in less than 1 second and preferably instantaneously. The rapid decrease in pressure results in the biomass separating into individual fibres or bundles of fibres. This step opens the fibre structure and increases the surface area. The lignin remains in the fibre along with cellulose and residual hemicellulose, which is then subjected to enzymatic hydrolysis for recovery of fermentable sugars from this residual cellulose and hemicellulose.

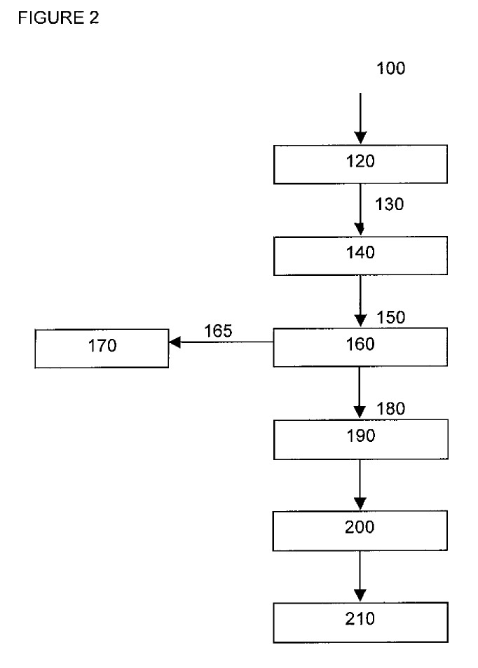

FIG. 2 exemplifies one embodiment of the invention that includes activation of the feedstock using autohydrolysis. Referring to FIG. 2, a lignocellulosic feedstock 100 is fed into a water and heat impregnator 120, where water and/or catalyst may be added to the feedstock. The addition of water is preferably carried out without steam addition to avoid the random and uncontrollable addition of moisture. The feedstock may be assayed for moisture content in order to carefully control the amount of amount water added to the feedstock. In a preferred embodiment, the moisture content of the feedstock is from about 45 to about 55% by weight before the start of autohydrolysis. The moist feedstock 130 is then subject to autohydrolysis in a hydrolyser 140. In some embodiments, the water and heat impregnation step can be performed in the same vessel as the hydrolyser.

The resulting autohydrolysed feedstock 150 may enter a solid/vapor separation unit 160, preferably a cyclone, to produce a vapor stream 165 and a solid stream 180. Separation unit 160 is preferably operated at vacuum to remove acetic acid, furfural and other volatile compounds. The vapor stream 165 may be passed to a scrubber 170 to remove volatile products, including water, some of which may be recycled.

Still referring to FIG. 2, the resulting autohydrolyzed solid stream 180 is then preferably subjected to disc refining 190 prior to enzymatic hydrolysis 200 and fermentation 210. Any disc refiner known in the art may be used. The applicants have found that passing the chemically hydrolyzed lignocellulosic feedstock through a disc refiner further activates the feedstock and increases the susceptibility of the feedstock to enzymatic hydrolysis. The use of a disc refiner also reduces the size of the particles in the feedstock as well as increasing the total available surface area of the particles in the feedstock.

The temperature in the disc refiner is preferably maintained at less than 65° C. Above this temperature, sugar degradation may occur decreasing the sugar content in the material. Preferably, the moisture content of the fiber passing through the disc refiner is about 50 to about 99% by weight.

The applicants have found that a disc refiner can be used with a lignocellulosic feedstock at a range of different particle sizes. Preferably, the size of the particles is from 0.0625 to 2 inches, more preferably 0.125 to 1 inch and most preferably 0.125 to 0.5 inches.

The optional use of a disc refiner prior to enzymatic hydrolysis is considered to enhance the conversion of cellulose to glucose and xylans to xylose. The use of a pulp disk refiner on an auto-hydrolyzed feedstock prior to enzymatic hydrolysis may result in an increase in the yield ratios of cellulose to glucose and xylans to xylose from about 60 to about 80% when a disc refiner is not used, to about 80 to about 95% when a disc refiner is used.

### First Enzymatic Hydrolysis Step

The feedstock after being subjected to any desired pretreatment is then subjected to enzymatic hydrolysis under vacuum. Any enzymatic hydrolysis process known in the art may be used. Preferably a two stage enzymatic hydrolysis is used.

The applicants herein describe a preferred optional method for efficiently breaking down a lignocellulosic feedstock into fermentable sugars.

Lignocellulosic feedstocks generally comprise cellulose, hemicellulose and lignin and have a high degree of polymerization. Hemicellulose is covalently linked to lignin, which in turn may be cross-linked to other polysaccharides such as cellulose resulting in a matrix of lignocellulosic material. Lignin is a hydrophobic cross-linked aromatic polymer and one of the major constituents of the cell walls of plants representing about one-quarter to one-third of the dry mass of wood.

Hemicellulose is a branched heteropolymer with a random, amorphous structure that includes a number of different sugar molecules such as xylose and arabinose. Xylose is the most common sugar molecule present in hemicellulose. Xylose and arabinose are both pentosans, which are polymeric 5-carbon sugars present in plant material.

Hemicellulase enzymes break down the hemicellulose structure. The use of hemicellulase enzymes results in the breakdown of the xylan backbone and side chains into pentosans such as xylose and arabinose as well as other sugars and polysaccharides. It will be apparent to those skilled in the art that most commercial preparations of hemicellulase enzyme also possess cellulase activity. Therefore, the first enzyme preparation (i.e. a hemicellulase enzyme preparation) used in the present disclosure, may possess about 10% to about 90% hemicellulase activity, preferably about 30% to about 90% hemicellulase activity, and more preferably about 50% or more (e.g. to about 90%) hemicellulase activity. In an embodiment, the hemicellulase preferentially acts upon the β-1,4 linkage of the xylose residues of xylan and the β-1,4 linkage of the mannose residues of mannan

Cellulose is a linear polymer of glucose wherein the glucose residues are held together by beta (1→4) glycosidic bonds. Cellulase enzymes catalyse the hydrolysis of cellulose into smaller polymeric units by breaking beta-glycosidic bonds. Endo-cellulase enzymes generally cleave internal glycosidic bonds in cellulose to create smaller polysaccharide chains, while exo-cellulase enzymes are able to cleave off 2-4 units of glucose from the ends of cellulose chains. Cellulase enzymes are not generally capable of cleaving cellulose into individual glucose molecules.

In contrast, cellobiase or beta-glucosidase enzymes catalyze the hydrolysis of a beta-glycosidic linkages in resulting in the release of at least one glucose molecule. Beta-glucosidase is therefore able to cleave cellobiose, which consists of two molecules of glucose joined together by a beta-glycosidic bond.

A person skilled in the art will appreciate that enzymes may exhibit a range of different activities on different substrates. As used herein, it is preferred that an enzyme preparation “preferentially acts” on a substrate when the relative activity of the enzyme for that substrate is greater than for other possible substrates. For example, a hemicellulase would preferentially act on hemicellulose to produce pentosans relative to its activity for cellulose to produce glucose.

An enzyme preparation may be a single enzyme or a combination of multiple enzymes. While enzyme preparations may be isolated from a number of sources such as natural cultures of bacteria, yeast or fungi a person skilled in the art will appreciate using enzymes produced using recombinant techniques.

In some embodiments, the applicants have found that the two-stage enzymatic hydrolysis process described in the present application is able to increase the sugar content of the resulting process stream which means starting with a high total solids content in the two-stage enzymatic hydrolysis.

As used herein, “total solids content” refers to the total amount of soluble and insoluble material in the feedstock. For example, in a lignocellulosic feedstock, soluble material would include monomeric sugars, some oligosaccharides, organic acids, extractives and low molecular weight compounds resulting from the autohydrolysis. Insoluble materials would include cellulose, lignin and hemicellulose. Suspensions with a high content of insoluble materials are generally difficult to process due to their high viscosity. Further, high-viscosity mixtures are difficult, if not impossible, to mix or handle through conventional pumping processes. In some embodiments, the sugar rich process stream described in the present application has a total solids content of greater than about 15%. In a further embodiment, the sugar rich process stream has a total solids content from about 15 to about 30%. In a further embodiment, the sugar rich process stream may have a total solids content up to about 50% (e.g., about 15 to about 50%, preferably about 30 to about 50%).

While not limited by a particular theory, the applicants note that by performing the enzymatic hydrolysis in two stages, the hemicellulase enzymes and in particular xylanase are not exposed to inhibitory concentrations of sugar monomers and dimers, and in particular glucose and cellobiose, that are produced during the second enzymatic hydrolysis stage.

The first enzymatic hydrolysis stage uses a first enzyme preparation that comprises hemicellulase. As will be known by those skilled in the art, the hemicellulase preparation will also possess cellulase activity. In one embodiment, the first enzyme preparation is a xylanase enzyme cocktail such as Dyadic XBP™. In a further embodiment, the first enzyme preparation is an enzyme cocktail such as AlternaFuel 100L™. It will be understood by a person skilled in the art that combinations of the enzyme preparations may be used. In an embodiment, the first enzyme preparation will possess hemicellulase activity from about 10% to about 90% and cellulase activity from about 90 to about 10%. In another embodiment, the hemicellulase activity will be from about 30% to 90% and the cellulase activity will be from about 70% to about 10%. In another embodiment, the hemicellulase activity will be from about 50 to 90% and the cellulase activity will be from about 50% to about 10%.

It will be appreciated that the use of the vacuum to remove volatile inhibiting compounds, such as acetic acid, can control or assist in controlling the pH of the slurry. As the pressure of the vacuum is decreased, more acetic acid would be removed resulting in an increase of the pH of the slurry. In embodiment, the pH of the process may also be adjusted using an acid stream to lower pH, or a base stream to increase the pH, such that the pH of the feedstock is in a range suitable for enzymatic activity. In a preferred embodiment, the pH is adjusted to be between about 4.5 to about 6.0.

The temperature of the first enzymatic process may also be controlled. In one embodiment the temperature of the process adjusted to be between about 20 to about 70° C. In a further embodiment, the first enzymatic process is conducted between about 30 to about 70° C. The process may be cooled using indirect cooling water, or warmed using indirect steam heating or by other methods known in the art.

The result of the first enzymatic process on the feedstock is a low viscosity effluent stream that may comprise xylans, cellobiose, glucose, xylose, lignin, ash, and organic acids. The low viscosity effluent stream may a viscosity that is at least about 15% lower than that of the feedstock, preferably at least about 20% lower and more preferably at least about 50% lower. Generally, the action of the first enzyme preparation results in the production of short-chain polysaccharides (oligosaccharides) such as cellobiose but not of large quantities of individual glucose molecules. Without being bound by theory, this is thought to prevent the hemicellulase enzymes in the first enzyme preparation from being inhibited by glucose molecules.

In one optional embodiment, the first enzymatic process is performed under vacuum and results in a volatile components stream, which can be removed from the low viscosity effluent stream. In one embodiment, the volatile component stream includes at least one yeast, fungi, bacteria or enzyme inhibiting compound present during the first enzymatic hydrolysis process and the volatile component stream that is drawn off includes at least one inhibiting compound. In another embodiment, the inhibiting compound in the volatile component stream may contain water, acetic acid, furfural, formic acid, and any other volatile organic compounds.

In an embodiment of the disclosure, the enzymatic hydrolysis is performed under a slight vacuum. The vacuum may be from 700 to 50 mm Hg (i.e, the pressure in the vessel may be from 700 to 50 mm Hg). Preferably, the vacuum is less than about 600 mm Hg, more preferably less than about 100 mm Hg and most preferably less than about 50 mm Hg. Preferably, the maximum vacuum that is applied is about 4 mm Hg.

In an embodiment of the disclosure, the concentration of acetic acid in the mixture in the first stage hydrolysis reactor is maintained at or below, or is reduced to or below 0.4% (w/v), preferably less than 0.3%, and more preferably less than 0.2%.

In an embodiment of the disclosure, the concentration of furfural in the mixture in the first stage hydrolysis reactor is maintained at or below, or is reduced to or below 0.2% (w/v), preferably less than 0.1%, and more preferably less than 0.05%.

### First Recycle Stream

In one embodiment, a recycle stream comprising material from the first enzymatic hydrolysis process is obtained and at least a portion of that recycle stream is preferably passed through a disc refiner or some other means of physically modifying (e.g. size reducing) the feedstock and reintroduced into the first enzymatic hydrolysis process. In an embodiment, the portion of the recycle stream that is passed through a refiner is between about 10 to about 90%. In another embodiment, all of a recycle stream from the bottom of the first enzymatic process tank is removed and is passed through a disc refiner before being reintroduced to the top of the first enzymatic process tank. The recycle stream can be mixed with fresh feedstock in the refiner, or prior to being reintroduced to the first enzymatic process tank. Preferably at least a portion of each of the feedstock and the recycle stream are fed through the disc refiner and, more preferably all of the feedstock and at least a portion of the recycle stream are fed through the disc refiner.

### Second Enzymatic Hydrolysis Step

If a two stage enzymatic process is used, then in the second enzymatic hydrolysis process, the low viscosity effluent stream is treated with a second enzyme preparation to produce a sugar rich process stream high in fermentable sugars such as glucose. In an optional embodiment, the second enzymatic hydrolysis process is also performed under vacuum.

The second enzyme preparation preferably primarily includes cellulase activity. In another embodiment, the second enzyme preparation comprises beta-glucosidase activity to convert disaccharides and other small polymers of glucose into monomeric glucose. In a further embodiment, the second enzyme preparation also includes cellulase activity. In one embodiment, the second enzyme preparation is Novozym 188™, available from Novozymes™. In another embodiment, the second enzyme preparation is NS50073™.

In one embodiment, the pH of the second hydrolysis process is adjusted using one or more of an acid stream or a base stream and vacuum to draw off volatile inhibitory compounds, such that the pH of the feedstock slurry is in a range suitable for enzymatic activity. In a preferred embodiment, the pH is adjusted to be between about 4.5 to about 5.4. In an embodiment, the acid stream comprises any mineral acid. In another embodiment, the acid stream comprises nitric acid, sulphuric acid, phosphoric acid, acetic acid and/or hydrochloric acid. In an embodiment, the base stream comprises potassium hydroxide, sodium hydroxide, ammonium hydroxide, urea and/or ammonia.

The temperature of the second enzymatic process may also be controlled. In one embodiment the temperature of the process adjusted to be between about 30 to about 70° C. In a further embodiment, the second enzymatic process is conducted between about 20 to about 70° C. The process may be cooled using indirect cooling water, or warmed using indirect steam heating or by other methods known in the art.

The resulting sugar rich process stream contains between about 5 to about 45% w/w fermentable sugars. Optional ranges include about 5 to about 30%, preferably about 10 to about 30% and more preferably about 15 to about 25%, as well as about 10 to about 45%, preferably about 15 to about 45% and more preferably about 25 to about 45%. The sugar rich process stream optionally also contains a total solids content of between about 10% to about 60%.

In an embodiment of the disclosure, the hydrolysis process is performed under a vacuum of less than about 700 mm Hg. In a further embodiment, the hydrolysis process is performed under a vacuum of less than about 50 mm Hg.

In an embodiment of the disclosure, the concentration of acetic acid in the mixture in the second stage hydrolysis reactor is maintained at or below, or is reduced to or below 0.4% (w/v), preferably less than 0.3%, and more preferably less than 0.2%.

In an embodiment of the disclosure, the concentration of furfural in the mixture in the second stage hydrolysis reactor is maintained at or below, or is reduced to or below 0.2% (w/v), preferably less than 0.1%, and more preferably less than 0.05%

### Vacuum

The presence of certain compounds in the lignocellulosic feedstock has been found by the applicants to have an inhibitory effect on enzymatic hydrolysis and on the fermentation of the resulting sugar streams. As used herein, “inhibiting compounds” are compounds that have an inhibitory effect on the enzymatic hydrolysis process, yeast fermentation or recovery of alcohols from lignocellulosic feedstocks. Examples of inhibiting compounds include furfural, hydroxymethylfurfural, organic acids, and phenolic compounds. In a further embodiment, the inhibitory compounds are furfural, acetic acid or formic acid. Preferably, the inhibiting compound is at least one of furfural and acetic acid. Other compounds and/or molecules that are also removed include nitrogen, oxygen, argon and carbon dioxide.

The applicants have found that performing the enzymatic hydrolysis of a lignocellulosic feedstock under vacuum allows for the removal of at least a portion of the inhibiting compounds from the feedstock or produced during the activation or the enzymatic hydrolysis. If a single stage enzymatic hydrolysis process is used, then this single stage may be conducted under vacuum. Alternately, if a multi-stage enzymatic hydrolysis process is used, then any one or more, and preferably all, of the stages are conducted under vacuum. The enzymatic hydrolysis steps are performed under vacuum so as to obtain a sugar rich process stream and a volatile components stream. In one embodiment, the volatile components stream is continuously removed from the first enzymatic hydrolysis process. In a preferred embodiment, the volatile components stream is removed by performing the enzymatic hydrolysis under vacuum pressure.

The applicants have also found that transferring the autohydrolyzed feedstock to a solid/vapor separation unit under vacuum pressure, preferably a cyclone, also results in removal of a volatile component stream. In a further embodiment, the fermentation step is also performed under vacuum pressure to remove inhibiting compounds. Accordingly, inhibitory compounds maybe removed by one or more steps, and in particular, prior to and/or during the enzymatic hydrolysis, and preferably at least during enzymatic hydrolysis.

In an embodiment of the disclosure, the enzymatic hydrolysis is performed under a slight vacuum. The vacuum may be from 700 to 50 mm Hg (i.e, the pressure in the vessel may be from 700 to 50 mm Hg). Preferably, the vacuum is less than about 600 mm Hg, more preferably less than about 100 mm Hg and most preferably less than about 50 mm Hg. Preferably, the maximum vacuum that is applied is about 4 mm Hg.

### OTHER EMBODIMENTS

In some embodiments, the sugar rich process stream is used to produce other sugar derived products. In one embodiment of the invention, the sugar rich process stream is used to produce alcohol through fermentation, preferably under vacuum pressure. The fermentable sugars such as glucose and xylose may be fermented to alcohol after yeast addition. In an embodiment, the alcohol produced is methanol, ethanol and/or butanol.

It will be appreciated that certain features of the invention, which are, for clarity, described in the context of separate embodiments or separate aspects, may also be provided in combination in a single embodiment. Conversely, various features of the invention, which are, for brevity, described in the context of a single embodiment or aspect, may also be provided separately or in any suitable sub-combination.

Although the invention has been described in conjunction with specific embodiments thereof, if is evident that many alternatives, modifications and variations will be apparent to those skilled in the art. Accordingly, it is intended to embrace all such alternatives, modifications and variations that fall within the spirit and broad scope of the appended claims. In addition, citation or identification of any reference in this application shall not be construed as an admission that such reference is available as prior art to the present invention.

### EXAMPLES

The operation of the invention is illustrated by the following representative examples, As is apparent to those skilled in the art, many of the details of the examples may be changed while still practicing the disclosure described herein.

### Example 1

### Vacuum Removal of Acetic Acid and Furfural at 50° C. and a pH of 3.4

Crushed poplar was pre-treated at 205° C. for 5.5 minutes in the SunOpta™ hydrolyzer. To the polar in the vessel was added 0.6 L of water to form a slurry of about 17% consistency. The reaction vessel was then closed and the slurry was mixed. After 5 minutes, the vessel was opened to ensure proper mixing was occurring. The reaction vessel was then closed and the slurry was mixed for 40 minutes. The reaction vessel was then connected to a vacuum pump resulting in a vacuum of about 20 to 17 mm Hg. After 10 minutes, the vacuum was closed and the condensate was recovered and was weighed an analyzed. The vacuum pump was then connected to the vessel and condensate samples were collected at time 20, 30,45, 69 and 90 minutes and each sample was analyzed.

### Example 2

### Vacuum Removal of Acetic Acid and Furfural at 50° C. and a pH of 5.0

Crushed poplar was pre-treated at 205° C. for 5.5 minutes in the SunOpta™ hydrolyzer. A reaction vessel was heated to temperature of about 50° C., and 0.4 kg of pre-treated poplar (wet basis) was added to the vessel. To the polar in the vessel was added 0.62 L of water to form a slurry of about 16.5% consistency. To this slurry, was added 21 g of 20% sodium hydroxide to adjust the pH to 5. The reaction vessel was then closed and the slurry was mixed. After 5 minutes, the vessel was opened to ensure proper mixing was occurring. The reaction vessel was then closed and the slurry was mixed for 40 minutes. The reaction vessel was then connected to a vacuum pump resulting in a vacuum of about 18 to 17 mm Hg. After 10 minutes, the vacuum was closed and the condensate was recovered and was weighed an analyzed. The vacuum pump was then connected to the vessel and condensate samples were collected at time 20, 30, 45, 69 and 90 minutes and each sample was analyzed.

### Example 3

### Vacuum Removal of Acetic Acid and Furfural at 60° C. and a pH of 3.4

The process in Example 1 was repeated, except that the temperature of the slurry being maintained at 60° C.

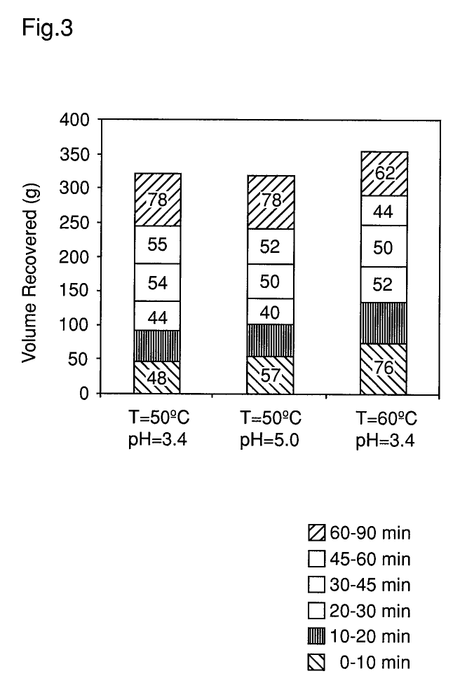

As exemplified in FIG. 3, which shows a graph of the volumes of water recovered from the pre-treated poplar for Examples 1, 2 and 3, only the reaction carried out at 60° C. resulted in a higher amount of condensate, which may be due to the drying of the slurry at the higher temperature. As seen, more than 350 g of water is removed from the slurry at a temperature of 60° C. at a pH of 3.4.

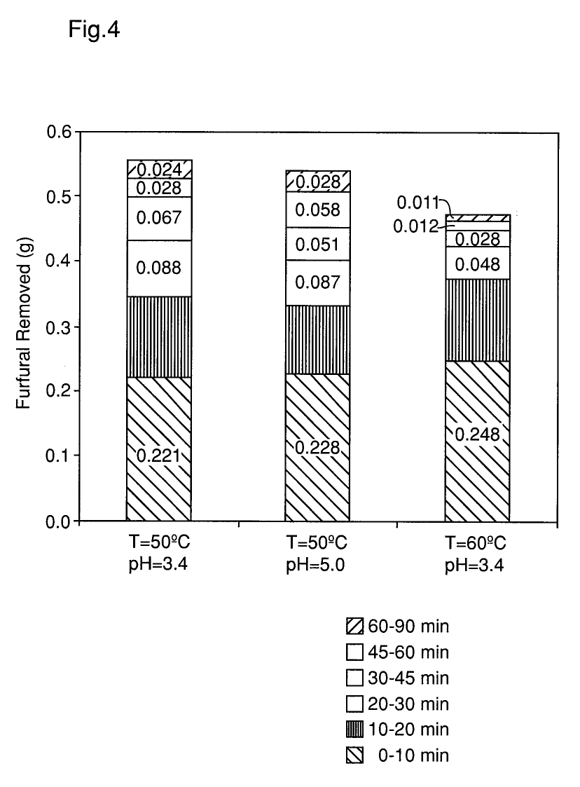

FIG. 4 shows a graph of the amount of furfural recovered from the pre-treated poplar slurry. The amount of furfural was not affected by the pH of the slurry, and less furfural was removed at the higher temperature of 60° C. It is possible that less furfural is removed from the slurry at higher temperatures because the mixing of the slurry becomes limited as a result of more water being evaporated. As seen in FIG. 4, the amount of furfural removed from the slurry approaches 0.6 g, with more than 0.2 g of furfural removed during the first 10 minutes at a temperature of 50° C. at a pH of 3.4.

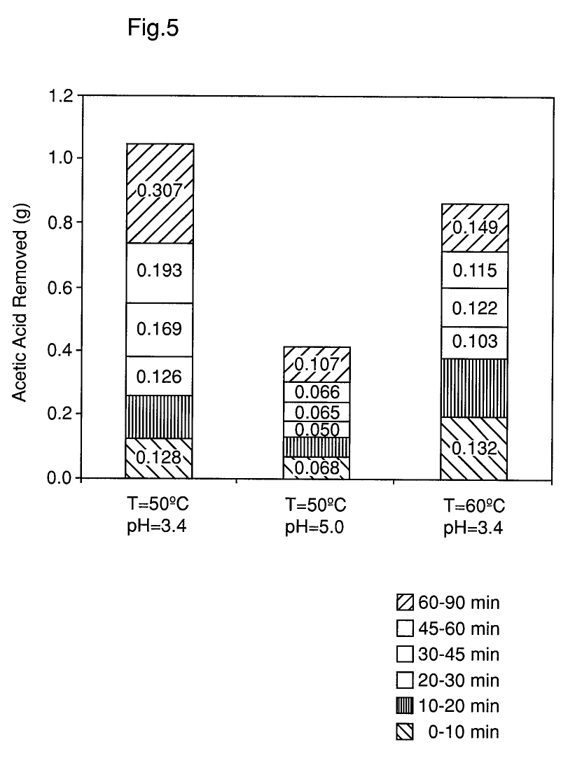

FIG. 5 shows a graph of the amount of acetic acid recovered from the pre-treated poplar, which was strongly affected by the pH of the slurry. As demonstrated in FIG. 5, the increase in the pH of the slurry to 5.0, resulted in a 60% decrease in the removal of acetic acid compared to a pH of 3.4. While less acetic acid is removed at 60° C., the acetic acid is removed at a faster rate during the first 20 minutes. As seen in FIG. 5, the amount of acetic acid removed from the slurry is more than 1.0 g at a temperature of 50° C. at a pH of 3.4.

In all cases, acetic acid and furfural were removed from the slurry when the vacuum was applied.

1. A method for treating a lignocellulosic feedstock comprising cellulose, hemicellulose and lignin to produce a sugar rich process stream, the method comprising subjecting the feedstock to enzymatic hydrolysis under vacuum and obtaining a volatile components stream and a sugar rich process stream.

2. The method of claim 1, wherein the enzymatic hydrolysis is performed under a vacuum pressure of less than about 700 mm Hg.

3. The method of claim 2, wherein the enzymatic hydrolysis is performed under a vacuum pressure of less than about 50 mm Hg.

4. The method of claim 1 wherein the feedstock is subjected to at least one of activation, extraction, hydrolysis and physical modification prior to enzymatic hydrolysis.

5. The method of claim 4 wherein the at least one of activation, extraction, hydrolysis and physical modification is produced by at least one of auto-hydrolysis, acid hydrolysis, ammonia activation, disc refining, kraft pulping, organic solvent pulping, hot water pretreatment, ammonia percolation, lime pretreatment, caustic solvent pulping and alkali peroxide pretreatment.

6. The method of claim 5 wherein the at least one of activation, extraction, hydrolysis and physical modification is autohydrolysis.

7. The method of claim 6 wherein after autohydrolysis, the feedstock is transferred to a cyclone under vacuum and a solid activated feedstock stream is obtained.

8. The method of claim 1 wherein the enzymatic hydrolysis comprises first and second enzymatic hydrolysis processes.

9. The method of claim 1 wherein the volatile components stream includes at least one yeast, fungi, bacteria and enzyme inhibiting compound comprising at least one of furfural, hydroxymethylfurfural, organic acids, phenolic compounds and other extractives.

10. The method of claim 9, wherein the concentration of furfural in the sugar rich process stream is less than about 0.2% (w/v).

11. The method of claim 10, wherein the concentration of furfural in the sugar rich process stream is less than about 0.1% (w/v).

12. The method of claim 11, wherein the concentration of furfural in the sugar rich process stream is less than about 0.05% (w/v).

13. The method of claim 9, wherein the organic acid is acetic acid or formic acid.

14. The method of claim 13, wherein the concentration of acetic acid in the sugar rich process stream is less than about 0.4% (w/v).

15. The method of claim 14, wherein the concentration of acetic acid in the sugar rich process stream is less than about 0.2% (w/v).

16. The method of claim 14, wherein the concentration of acetic acid in the sugar rich process stream is less than about 0.1% (w/v).

17. The method of claim 8 wherein the method further comprises operating the first enzymatic hydrolysis process under a sufficient vacuum to reduce the level of the at least one inhibiting compound.

18. The method of claim 17 wherein the first enzymatic hydrolysis is operated under a vacuum of at least about 700 mm Hg.

19. The method of claim 18 wherein the first enzymatic hydrolysis is operated under a vacuum of at least about 50 mm Hg.

20. The method of claim 8 wherein the method further comprises operating the second enzymatic hydrolysis under a sufficient vacuum to reduce the level of the at least one inhibiting compound.

21. The method of claim 8 wherein the first enzymatic hydrolysis process uses a first enzyme preparation and produces a volatile component stream and a low viscosity effluent stream and the low viscosity effluent stream is subjected to the second enzymatic hydrolysis using a second enzyme preparation and produces the sugar rich process stream.

22. The method of claim 21 wherein the first enzyme preparation has hemicellulase activity and cellulase activity.

23. The method of claim 22 wherein the first enzyme preparation has a hemicellulase activity from about 10 to about 90% and a cellulase activity from about 90 to about 10%.

24. The method of claim 23 wherein the first enzyme preparation preferentially acts upon the hemicellulose relative to cellulose in the feedstock.

25. The method of claim 24 wherein the first enzyme preparation comprises hemicellulase and cellulase enzymes, wherein the hemicellulase enzymes preferentially act upon the β-1,4 linkage of the xylose residues of xylan and the β-1,4 linkage of the mannose residues of mannan.

26. The method of claim 21 wherein the second enzyme preparation preferentially acts on the cellulose and cellobiose relative to xylan in the feedstock.

27. The method of claim 26 wherein the second enzyme preparation comprises β-glucosidase and cellulase enzymes, wherein the β-glucosidase and cellulase enzymes preferentially act upon the β-1,4 linkage of cellobiose and cellulose.

28. The method of claim 27 wherein the β-glucosidase and cellulase enzymes completely convert cellulose and oligosaccharides produced from the first enzymatic hydrolysis to monomeric sugars.

29. A method for the production of an alcohol from a lignocellulosic feedstock, comprising:
(a) pretreating the feedstock;
(b) transferring the pretreated feedstock to a cyclone under vacuum;
(c) subjecting the feedstock from the cyclone to an enzymatic hydrolysis under vacuum to produce a sugar rich process stream; and,
(d) fermenting the sugar rich process stream under vacuum to obtain the alcohol.

30. The method according to claim 29 wherein the feedstock is pretreated by at least one of activation, extraction, hydrolysis and physical modification.

31. The method according to claim 30 wherein the at least one of activation, extraction, hydrolysis and physical modification is produced by at least one of auto-hydrolysis, acid hydrolysis, ammonia activation, disc refining, kraft pulping, organic solvent pulping, hot water pretreatment, ammonia percolation, lime pretreatment, caustic solvent pulping, and alkali peroxide pretreatment.

32. The method according to claim 31 wherein the at least one of activation, extraction, hydrolysis and physical modification is autohydrolysis.

33. The method of claim 32 wherein after autohydrolysis, the feedstock is transferred to a cyclone under vacuum and a solid activated feedstock stream is obtained.

34. The method of claim 29 wherein the enzymatic hydrolysis comprises first and second enzymatic hydrolysis, wherein the first enzymatic hydrolysisis performed under a vacuum.

35. The method of claim 34 wherein the second enzymatic hydrolysis is performed under a vacuum.
